(19) United States
(12) Patent Application Publication
(10) Pub. No.: US 2008/0229804 A1
(43) Pub. Date: Sep. 25, 2008

(54) GAS SENSOR

(75) Inventor: Masanobu YAMAUCHI, Kariya-shi (JP)

Correspondence Address: NIXON & VANDERHYE, PC, 901 NORTH GLEBE ROAD, 11TH FLOOR, ARLINGTON, VA, 22203, US

(73) Assignee: DENSO CORPORATION, Kariya-city (JP)

(21) Appl. No.: 12/044,124

(22) Filed: Mar. 7, 2008

(30) Foreign Application Priority Data
Mar. 21, 2007 (JP) 2007-073776

(51) Int. Cl. G01N 33/22

(52) U.S. Cl. 73/23.31

(57) ABSTRACT

A gas sensor has a sensor element, a housing, an atmosphere-side cover, lead wirings, an elastic member, and a filter assembly. The elastic member has a vertical-hole and lead-wiring holes. The vertical-hole penetrates the elastic member in an axial direction of the gas sensor. The lead wirings are disposed in the lead-wiring holes. The filter assembly composed of a support member and an air filter is fitted to the vertical-hole of the elastic member. The air filter covers at least an end-side opening part and an outer periphery surface connected to the opening part of the support member. The atmosphere-side cover has fastening parts fastening the elastic member. A part of the air filter covering the outer periphery surface of the support member is fastened between the elastic member and the support member. A part of the air filter is disposed at the inside of the support member.

### CROSS-REFERENCE TO RELATED APPLICATION

This application is related to and claims priority from Japanese Patent Application No. 2007-73776 filed on Mar. 21, 2007, the contents of which are hereby incorporated by reference.

### BACKGROUND OF THE INVENTION

1. Field of the invention

The present invention relates to a gas sensor capable of detecting a concentration of a specific gas component contained in a target gas.

2. Description of the Related Art

There is a gas sensor capable of detecting or measuring a concentration of oxygen gas contained in an exhaust gas, which is assembled into an exhaust gas system for an internal combustion engine of a motor vehicle.

FIG. 13 is a sectional view of a gas sensor according to a related art. As shown in FIG. 13, a gas sensor 9 has a built-in gas sensor element 910 capable of detecting a concentration of a specific gas (such as oxygen gas) contained in a target gas (such as an exhaust gas) emitted from an internal combustion engine of a motor vehicle.

The gas sensor element 910 is inserted into and fitted to a housing 911 through an insulation glass 913. An atmosphere-side cover 92 is fixed to a base end side of the housing 911. As shown in FIG. 13, an elastic member 93 is disposed at the base end side of the atmosphere-side cover 92, with which the base end side thereof is sealed. Lead wirings 912 are electrically connected to the gas sensor element 910 and placed through the elastic member 93.

An outer periphery cover 94 is fixed to a base end side of the atmosphere-side cover 92. The outer periphery cover 94 is fastened toward the inside of a radial direction of the gas sensor 9 at three fastening parts 940 which are formed along the axial direction of the gas sensor 9.

An air filter 95 having a water-proof function or property is supported between the atmosphere-side cover 92 and the outer periphery cover 94, and fastened at two points which are formed in the axial direction of the gas sensor 9.

The atmosphere gas as a reference gas is introduced from a penetration hole 941 formed in the outer periphery cover 94 into an air inlet formed in the atmosphere-side cover 92 through an air filter 95. The atmosphere gas is then introduced in the inside of the gas sensor 9.

However, the related art gas sensor 9 shown in FIG. 13 has a following drawback. Because the air filter 95 is, as described above, disposed between the atmosphere-side cover 92 and the outer periphery cover 94, heat energy is easily propagated into the air filter 95 of the water-proof function through the atmosphere-side cover 92 or the outer periphery cover 94. Repetitive use of the gas sensor 9 in an exhaust gas system for an internal combustion engine thermally deteriorates the quality of the water-proof air filter 95, and further decreases the water-proof function of the fastening parts 940 for fastening the air filter 95 having a water-proof function. As a result, there is a possibility that outer water enters or permeates the inside of the gas sensor 9.

In order to avoid such a related art drawback, Japanese patent laid open publication No. JP 2000-249678 has disclosed a gas sensor 8 in which a support member 84 is disposed in a vertical hole 830 formed in an elastic member 83 in the gas sensor 8, where a base part of the support member 84 is covered with a sheet-like air filter 85, as shown in FIG. 14.

Because the air filter 85 is supported by the elastic member 83 in the gas sensor 8 having the above configuration, it is possible to keep the adhesion between the air filter 85 and the elastic member 83 even if thermal deterioration such as a shrinkage of the air filter 85 occurs by heat energy of an exhaust gas.

However, the related art gas sensor 81 involves a following drawback. It is necessary to insert and place a sheet-like air filter 85 and a circular-shaped insert member 84 into the vertical hole 830 which is formed in the elastic member 83 while positioning them when an air passage for introducing the outer atmosphere as a target gas is formed in the gas sensor 8. It is thereby difficult to incorporate the air filter 85 into the gas sensor 8 and difficult to enhance or increase the manufacturing productivity of the gas sensor. Further, because the air filter 85 has a weak sheet-like member, the requested durability of the air filter 85 is difficult to meet.

### SUMMARY OF THE INVENTION

It is an object of the present invention is to provide a gas sensor having an air passage part such as an air filter which does not deteriorate any water proof function, capable of preventing decreasing a water-proof function caused by the passage of time, with high manufacturing productivity and durability.

To achieve the above purposes, the present invention provides a gas sensor which has a sensor element, a housing, lead wirings, an elastic member, an atmosphere-side cover, and a filter assembly. The sensor element is configured to detect a concentration of a specific gas component contained in a target gas. The housing accommodates the sensor element so that the sensor element is fitted to and supported by the housing. The lead wirings are electrically connected to the sensor element. The elastic member has a vertical hole in an axis direction of the gas sensor and lead wiring penetration holes, through which the lead wirings are inserted and placed. The elastic member seals the base end side of an atmosphere-side cover. The atmosphere-side cover is disposed at a base end side of the housing. The atmosphere-side cover has fastening parts at which the elastic member is fastened toward a radius direction of the gas sensor. The filter assembly is comprised of a cylindrical shaped support member and an air filter. The air filter is fitted to the cylindrical shaped support member so that the air filter covers at least a base end side opening part of the cylindrical shaped support member and a part of an outer periphery surface continued to the base end side opening part of the cylindrical shaped support member, a part of the air filter covering the outer periphery surface of the support member is fitted and pressed between the elastic member and the support member, and a part of the air filter is disposed in the inside of the cylindrical shaped support member.

The gas sensor according to the present invention has the following superior actions and effects.

In the gas sensor according to the present invention, the filter assembly is composed of the cylindrical shaped support member and the air filter and inserted into and fitted to the vertical hole formed in the elastic member. That is, because the filter assembly composed of the support member and the air filter is supported, it is possible to easily assemble the filter assembly to the gas sensor. As a result, the manufacturing process has been made easier through the gas sensors configuration.

Because the air filter has the above configuration, it is possible to form the air filter of some thickness which is thicker than a sheet-like air filter and thereby easily maintain the strength state of the air filter in the gas sensor when compared with a gas sensor having a sheet-like air filter. This configuration can provide the gas sensor with high durability.

Further, according to the present invention, because the inside filter part, as a part of the air filter, is disposed in the inside of the support member, the elastic member part does not directly push the inside filter part. It is thereby possible to prevent the deformation and breaking of the inside filter part, namely, to break pores formed in the inside filter part. This can certainly keep the gas permeable function of the air filter for a long period of time.

In addition, according to the present invention, because the thickness of the air filter in the gas sensor can be increased toward the longitudinal direction of the support member, it is possible to increase the strength of the air filter.

Still further according to the present invention, the part of the air filter which covers the outer periphery surface of the support member is supported in such a way as to be pushed between the elastic member part and the support member. Therefore it is possible to keep or maintain the adhesive state between the air filter and the elastic member part even if deterioration of the air filter such as shrinkage occurs by heat energy when the gas sensor is used under harsh conditions, for example, in an exhaust gas passage in an exhaust gas system for an internal combustion engine of a motor vehicle.

That is, even if thermal or heat deterioration occurs in the air filter, the elastic stress or power of the elastic member part applied toward the support member can keep the adhesive state between the surface of the air filter and the surface of the elastic member part according to deformation such as shrinkage of the air filter. As a result, it is thereby possible to prevent any deterioration of the water-proof function of the air filter part, through which outer air atmosphere is introduced, over the passage of time. Thus, it is possible for the elastic stress or power of the elastic member part to compensate for the heat deterioration of the air filter.

Still further, according to the present invention, the air filter is supported by a vertical hole of the elastic member part. This means that the air filter is supported by an elastic member part which does not conduct thermal energy well. This can prevent the heat propagation from the atmosphere-side cover toward the inside of the gas sensor through the gas filter, and also prevent the progress of heat deterioration of the air filter.

According to the present invention, it is possible to provide the gas sensor with superior manufacturing efficiency and durability, capable of preventing the deterioration of its water proof function, over the passage of time.

The gas sensor according to the present invention can be applied to a A/F sensor, a NOx sensor, an oxygen sensor, and the like.

Through the specification, the front end part of the gas sensor is inserted and disposed in an exhaust gas flow in an exhaust gas passage for an internal combustion engine. The opposite of the front end part of the gas sensor is referred to as the “base-end part.”

In the gas sensor as another aspect of the present invention, the air filter is fitted to the base end side of the cylindrical shaped support member. Because the air filter and support member are easily assembled into a single body, it is easy to assemble the filter assembly to the gas sensor.

In the gas sensor as another aspect of the present invention, the air filter and the cylindrical shaped support member are made in one assembly. Because the filter assembly is easily formed, it is possible to increase the manufacturing efficiency of the gas sensor.

In the gas sensor as another aspect of the present invention, at least a base end side part of the cylindrical shaped support member is embedded in the air filter. Thereby, because workers can easily assemble the filter assembly, it is possible to provide the gas sensor with more superior manufacturing efficiency.

In the gas sensor as another aspect of the present invention, the air filter has a hollow part formed in the inside thereof, which faces the inside of the cylindrical shaped support member. In this case, it is possible to easily adjust the valance relationship between the air permeability and the strength of the air filter by adjusting the thickness of a part of the air filter, which is disposed in the inside of the support member, by adjusting the size and shape of the hollow part.

In the gas sensor as another aspect of the present invention, the cylindrical shaped support member is embedded in the air filter. According to the configuration of the gas sensor of the present invention, because workers can easily assemble the filter assembly, it is possible to provide the gas sensor with superior manufacturing efficiency.

In the gas sensor as another aspect of the present invention, the air filter is made of porous polytetrafluoroethylene (PTFE). Because the air filter is made of porous PTFE, the air filter has a superior water-proof function and a superior thermal proof function, and has a high resistance to chemicals. It is further possible to prevent the deterioration of its air-permeable function even if used under harsh conditions.

### BRIEF DESCRIPTION OF THE DRAWINGS

A preferred, non-limiting embodiment of the present invention will be described by way of example with reference to the accompanying drawings, in which:

FIG. 1 is a sectional view showing a gas sensor having a laminated or multilayer sensor element according to a first embodiment of the present invention;

FIG. 2 is a perspective view of a filter assembly built in the gas sensor according to the first embodiment of the present invention;

FIG. 3 is a sectional view of the filter assembly in the gas sensor according to the first embodiment of the present invention;

FIG. 4 is a sectional view of the gas sensor having a cup shaped sensor element according to a modification of the first embodiment of the present invention;

FIG. 5 is a sectional view of a filter assembly in a gas sensor according to a second first embodiment of the present invention;

FIG. 6 is a sectional view of the filter assembly having another configuration in the gas sensor according to the second embodiment of the present invention;

FIG. 7 is a sectional view of the filter assembly having another configuration in the gas sensor according to the second embodiment of the present invention;

FIG. 8 is a sectional view of a filter assembly in a gas sensor according to a third embodiment of the present invention;

FIG. 9 is a sectional view of the filter assembly having another configuration in the gas sensor according to the third embodiment of the present invention;

FIG. 10 is a sectional view of the filter assembly having another configuration in the gas sensor according to the third embodiment of the present invention;

FIG. 11 is a sectional view of a filter assembly in a gas sensor as a comparison example;

FIG. 12 is a sectional view of the filter assembly having another configuration in the gas sensor as the comparison example;

FIG. 13 is a sectional view of a gas sensor according to a related art; and

FIG. 14 is a sectional view showing a base end part of the gas sensor according to the related art.

### DETAILED DESCRIPTION OF THE PREFERRED EMBODIMENTS

Hereinafter, various embodiments of the present invention will be described with reference to the accompanying drawings. In the following description of the various embodiments, like reference characters or numerals designate like or equivalent component parts throughout the several diagrams.

### First embodiment

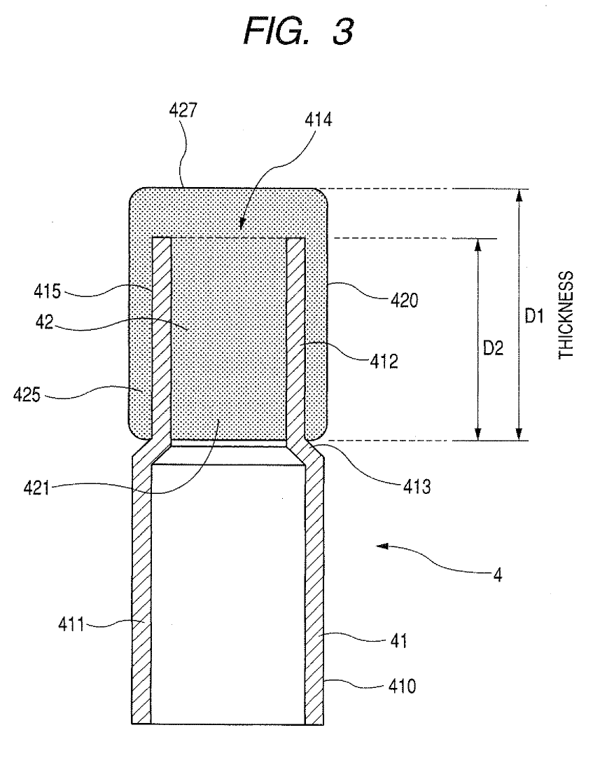

A description will be given of a gas sensor according to a first embodiment of the present invention with reference to FIG, 1 to FIG. 3.

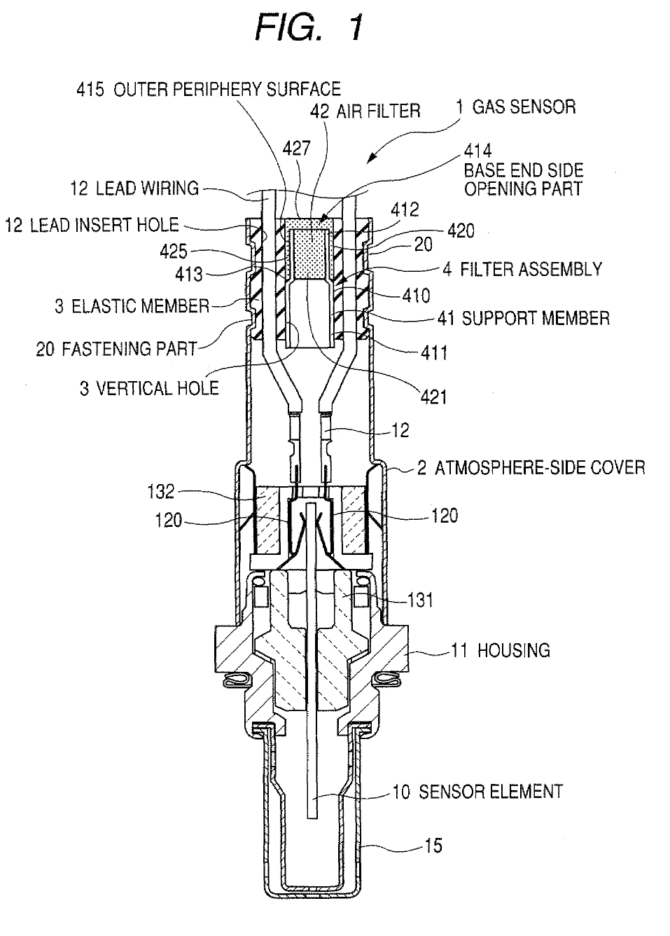

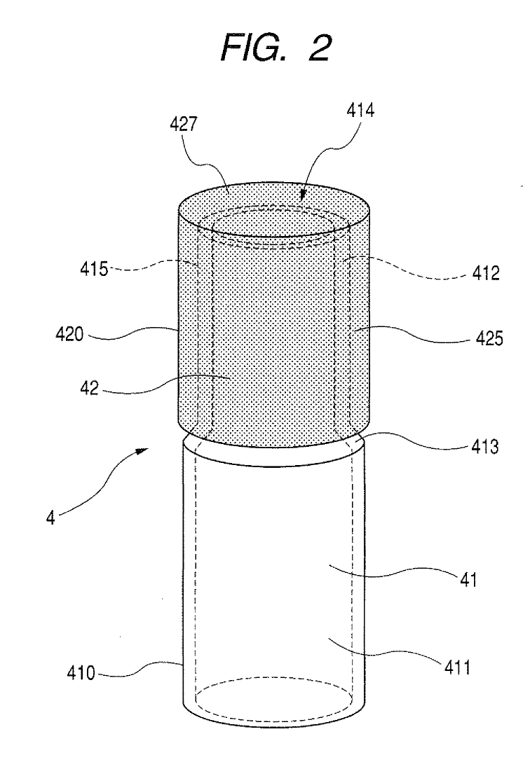

FIG. 1 is a sectional view showing a gas sensor 1 having a gas sensor element 10 as a laminated or multilayer sensor element according to the first embodiment of the present invention. FIG. 2 is a perspective view of a filter assembly in the gas sensor 1 shown in FIG. 1. FIG. 3 is a sectional view of the filter assembly in the gas sensor I shown in FIG. 1.

As shown in FIG. 1, the gas sensor 1 according to the first embodiment is comprised of a gas sensor element 10, a housing 11, an atmosphere-side cover 2, a pair of lead wirings 12, and an elastic member part 3. The gas sensor element 10 detects a concentration of a specific gas component contained in a target gas such as an exhaust gas emitted from an internal combustion engine mounted to a motor vehicle. The housing accommodates and supports the gas sensor element 10. The atmosphere-side cover 2 is disposed at a base end side of the housing 11. The lead wirings 12 are electrically connected to the gas sensor element 10. A base end part of the atmosphere-side cover 2 is sealed with the elastic member part 3. The lead wirings 12 are placed in the elastic member part 3.

As shown in FIG. 1, the elastic member part 3 has a vertical hole 30 which penetrates in the elastic member part 3 and lead wiring insert-holes through which the lead wirings 12 are inserted and placed therein.

As shown in FIG. 1, FIG. 2, and FIG. 3, a filter assembly 4 is inserted in and fitted to the vertical hole 30 in the gas sensor 1. The filter assembly 4 has a cylindrical shaped support member 41 and an air filter 42. In particular, as shown in FIG. 2 and FIG. 3, the air filter 42 is attached and fitted to the cylindrical shaped support member 41 so that the air filter 42 covers a part of an outer periphery surface 415 of the support member 41 connected to a base end side opening part 414 of the filter assembly 4.

As shown in FIG. 1, the atmosphere-side cover 2 has fastening parts 20 which fasten the elastic member part 3 toward the inside direction of the radial of the gas sensor 1. The elastic member part 3 in the gas sensor 1 according to the first embodiment is fastened at two parts which are formed along the axis direction (or a longitudinal direction) of the gas sensor 1.

A part 425 covering the outer periphery surface 415 of the cylindrical shaped support member 41 in the air filter 42 is fastened between the elastic member part 3 and the support member 41.

A description will now be given of the gas sensor 1 according to the first embodiment of the present invention.

As shown in FIG. 1, the sensor element 10 is inserted in and fastened to a target measurement gas side insulation glass 131. An atmosphere-side insulation glass 132 is disposed at the base end side of the housing 11 so that it covers the base end side of the sensor element 10. On the other hand, an element side cover 15 is disposed at a front end part of the housing 11 in order to protect the front part of the sensor element 10.

The gas element 10 has a detection part (not shown) and a heater part (not shown), and electric terminals (not shown). The detection part detects a concentration of a specific gas contained in a target gas such as an exhaust gas. The heater part heats the detection part. The electric terminals are electrically connected to the detection part and the heater part,

Each terminal is electrically connected to one of four lead wirings 12 through a connection terminal 120 disposed in the atmosphere-side insulation glass 132.

The lead wirings 12 are inserted into lead insert holes 32 formed in the elastic member part 3 at the base end part of the atmosphere-side cover 2. The lead wirings 12 are extended toward the outside of the gas sensor 1.

The atmosphere-side cover 2 is made of stainless steel, and as described above, has the fastening parts 20 capable of fastening the elastic member part 3 at two parts toward the inside of the radial direction of the gas sensor 1. That is, the elastic member part 3 is fastened at the fastening parts 20 of the atmosphere-side cover 2 toward the inside of the radial direction. The lead wirings 12 closely adhere to the lead insert holes 32 by the fastening parts 20.

As shown in FIG. 1, the vertical hole 30 is formed in the elastic member part 3 so that it penetrates the elastic member 3. The filter assembly 4 composed of the cylindrical shaped support member 41 and the air filter 42 is inserted into, and fitted into and supported by the vertical hole 30.

As shown in FIG. 1 to FIG. 3, the support member 41 is composed of a large radius part 411 and a small radius part 412 which is smaller in diameter than the large radius part 411. The large radius part 411 is disposed toward a front end side of the gas sensor 1. The small radius part 412 is disposed at the base end side of the air filter 1 when observed from the large radius part 411. A taper part 413 is formed between the large radius part 411 and the small radius part 412.

As shown in FIG. 1 to FIG. 3, the air filter 42 is fitted to the small radius part 412 of the cylindrical shaped support member 41. The air filter 42 is disposed in the entire of the inside of the small radius part 412 of the support member 41. That is, as shown in FIG. 3, a part of a thickness D2 (measured toward the longitudinal direction of the gas sensor 1) of the air filter 42 is completely fitted to the small radius part 412 of the cylindrical shaped support member 41.

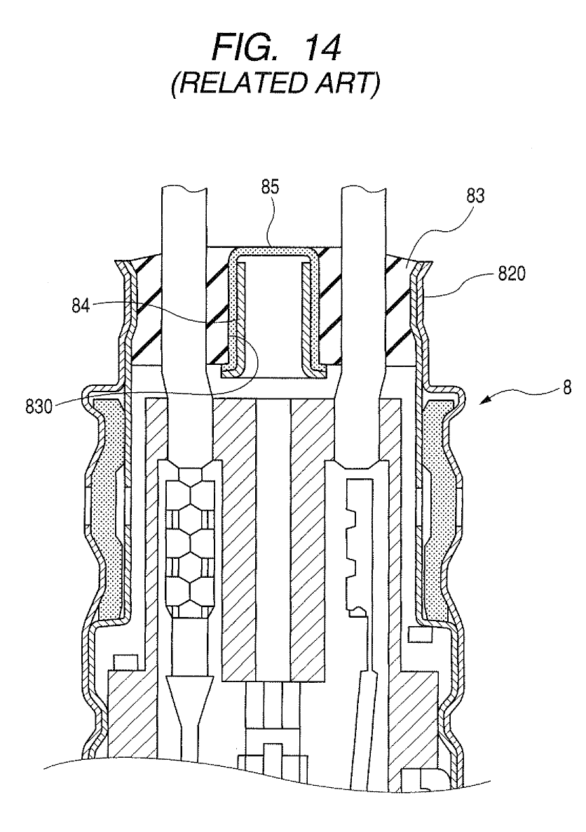

As described above and shown in FIG. I to FIG. 3, the air filter 42 in the gas sensor I according to the first embodiment of the present invention is a block shaped member, not a sheet-shaped member (for example, designated by reference number 85 in the related art shown in FIG. 14). Hereinafter, the part disposed in the inside of the cylindrical shaped support member 41 in the air filter 42 will be referred to as the “inside filter part 421” for short.

That is, as shown in FIG. 3, the air filter 42 is made of porous polytetrafluoroethylene (porous PTFE). The air filter 42 and the cylindrical shaped support member 41 are made in one assembly.

The air filter 42 and the large radius part 411 take approximately the same diameter. In other words, the outer periphery surface 420 of the air filter 42 and the outer periphery surface 410 of the large radius part 411 take approximately the same surface. It is thereby possible to form the vertical hole 30 in the elastic member part 3 as a straight hole because the cylindrical shaped support member 41 and the air filter 42 are certainly fitted and supported in the vertical hole 30 in the elastic member 3.

Next, a description will now be given of an air introduction passage in the gas sensor 1 according to the first embodiment with reference to FIG. 1.

Atmosphere gas such as air is introduced into the inside of the gas sensor 1 trough the base end part 427 of the air filter 42. Because the air filter 42 is made of porous PTFE, the air filter 42 easily introduces the atmosphere gas into the inside of the gas sensor 1, does not become an obstacle to introduction of the atmosphere gas from the outside of the gas sensor 1. That is, the atmosphere gas such as air is introduced into the cylindrical shaped support member 41 through the air filter 42. Because the support member 41 has a cylindrical shape, the air atmosphere is introduced into the atmosphere-side cover 2 through the inside of the support member 41.

The air atmosphere is then introduced into the sensor element 10, and finally reaches a reference gas space or room formed in the inside of the sensor element 10.

A description will now be given of the effects and actions of the gas sensor 1 according to the first embodiment of the present invention having the configuration described above.

As shown in FIG. 1, the filter assembly 4 composed of the cylindrical shaped support member 41 and the air filter 42 is inserted in and fitted to the vertical hole 30. That is, because the assembly composed of the support member 41 and the air filter 42 is supported, it is possible to easily assemble it to the gas sensor 1 of the first embodiment of the present invention. This configuration of the gas sensor 1 can provide an easy manufacturing process.

Because the air filter 42 has some thickness (designated by reference character D1 shown in FIG. 3) when compared with a sheet-like filter, it is possible to easily keep the strength of the air filter 42. This configuration can provide the gas sensor with superior durability.

Further, because the inside filter part 421, as a part of the air filter 42, is disposed in the inside of the support member 41, the elastic member part 3 does not directly push the inside filter part 421. It is thereby possible to prevent the deformation and breaking of the inside filter part 421, namely, breaking pores formed in the inside filter part 421. This can certainly keep the gas permeable function of the air filter 42 for a long period of time.

Still further, as shown in FIG. 1, the part 425 covering the outer periphery surface 415 of the support member 41 which supports the air filter 42 is supported in the state where the part 452 is pushed between the elastic member part 3 and the support member 41. Therefore it is possible to keep or maintain the adhesive state between the air filter 42 and the elastic member part 3 even if deterioration of the air filter, such as shrinkage occurs by heat energy when the gas sensor 1 is used under harsh conditions, for example, in an exhaust gas passage in an exhaust gas system for an internal combustion engine of a motor vehicle.

That is, even if the thermal or heat deterioration occurs in the air filter 42, the elastic stress or power of the elastic member part 3 applied toward the support member 41 can keep the adhesive state between both the surface of the air filter 42 and the surface of the elastic member part 3 according to deformation such as shrinkage of the air filter 42. As a result, it is thereby possible to prevent the deterioration of the water-proof function of the air filter part, through which outer air atmosphere is introduced, over the passage of time. Thus, it is possible for the elastic stress or power of the elastic member part 3 to compensate for the heat deterioration of the air filter 42.

Still further, as shown in FIG. 1, the air filter 42 is supported by the vertical hole 30 of the elastic member part 3. This means that the air filter 42 is supported by the elastic member part 3 having a small thermal conductivity. This can prevent the heat propagation from the atmosphere-side cover 2 toward the inside of the gas sensor 1 through the gas filter 42, and also prevent the progress of heat deterioration of the air filter 42.

Because the air filter 42 is fitted to the base end side of the support member 41, it is possible to easily assemble the air filter 42 the support member 41 into the filter assembly 4. It is thereby possible to easily assemble the filter assembly 4 into the gas sensor 1.

Still further, the air filter 42 and the support member 41 are made in one assembly. Because the filter assembly 4 is easily assembled, it is possible to easily provide the gas sensor 1 with superior manufacturing efficiency.

The small radius part 412 of the support member 41 is embedded in the air filter 42. It is thereby possible to more easily assemble the filter assembly 4. This configuration provides the gas sensor 1 with superior manufacturing efficiency. Because the thickness D1 (see FIG. 3) of the air filter 42 can be increased by inserting it toward the longitudinal direction of the support member 41, it is possible to increase the strength of the air filter 42.

Still further, because the air filter is made of porous PTFE, the air filter has a superior water-proof function and a superior thermal proof function, and has a high resistance to chemicals. It is thereby possible to provide the air filter 42 capable of preventing deterioration of the air permeable function even if used under various strict conditions.

As described above, according to the first embodiment of the present invention, it is possible to provide the gas sensor with high manufacturing efficiency, high durability, and capable of preventing the deterioration of its water proof function, over the passage of time.

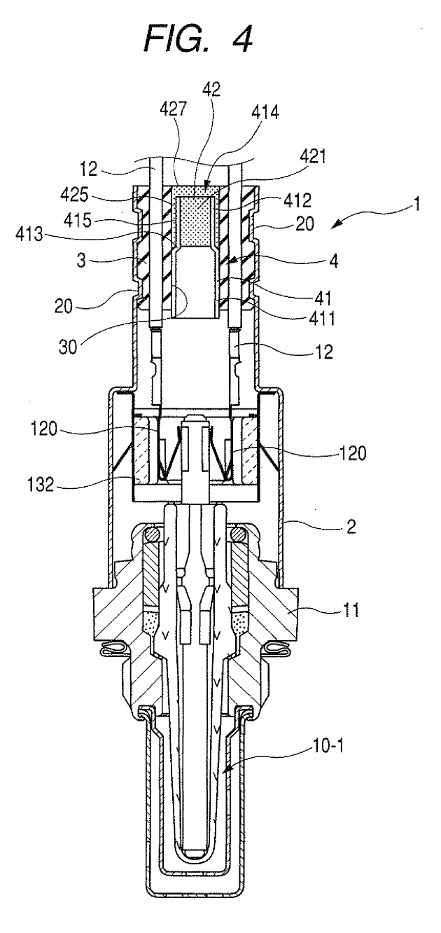

FIG. 4 is a sectional view of the gas sensor having a cup-shaped sensor element according to a modification of the first embodiment of the present invention. The gas sensor 1 according to the first embodiment described above is a multilayer type (or a lamination type) gas sensor. The present invention is not limited by this configuration. For example, as shown in FIG. 4, it is possible to apply the concept of the first embodiment of the present invention to a cup-shaped gas sensor having a sensor element 10-1. Other reference numbers in FIG. 4 are the same of those in FIG. 1.

### Second Embodiment

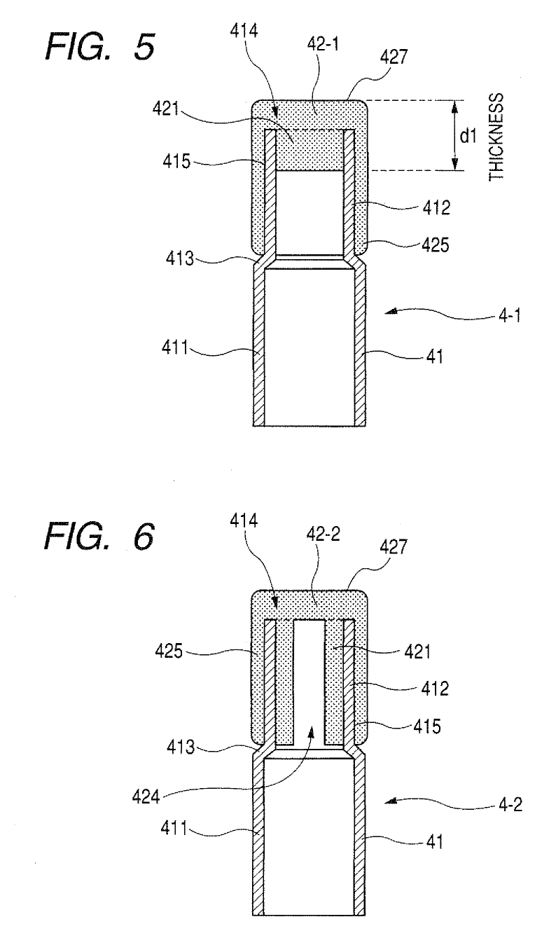

A description will be given of a gas sensor according to a second embodiment of the present invention with reference to FIG. 5 to FIG. 7.

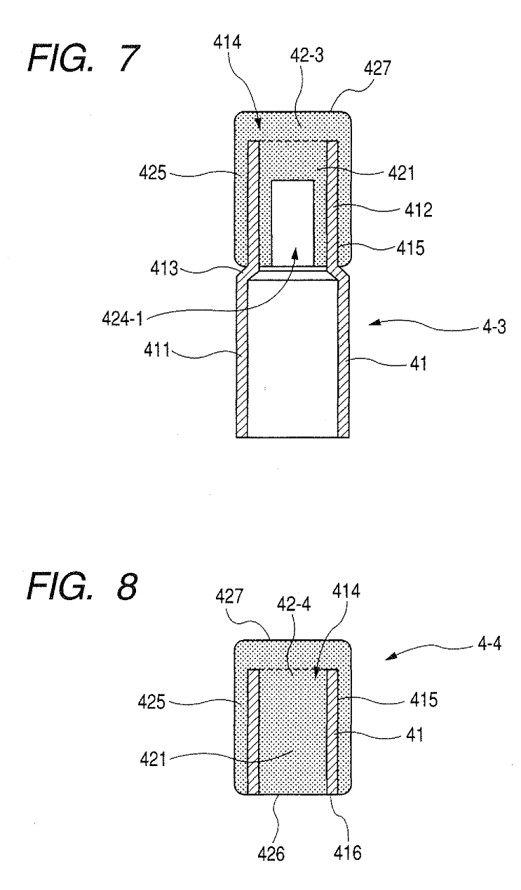

FIG. 5 is a sectional view of a filter assembly in the gas sensor according to the second embodiment. FIG. 6 is a sectional view of the filter assembly having another configuration in the gas sensor according to the second embodiment. FIG. 7 is a sectional view of the filter assembly having another configuration in the gas sensor according to the second embodiment.

As shown in FIG.5 to FIG. 7, the gas sensor according to the second embodiment has various types of filter assemblies 4-1 (see FIG. 5), 4-2 (see FIG. 6), and 4-3 (see FIG. 7) having a different shape.

As shown in FIG. 5, an air filter 42-1 in the filter assembly 4-1 in the gas sensor of the second embodiment has a thickness d1 which is smaller than the thickness D1 of the air filter 42 in the filter assembly 4 of the first embodiment shown in FIG. 2 and FIG. 3.

Further, as shown in FIG. 6, an air filter 42-2 in a filter assembly 4-2 in the gas sensor according to another configuration of the second embodiment has a hollow part 424 formed in the inside filter part 421.

Still further, as shown in FIG. 7, an air filter 42-3 in a filter assembly 4-3 in the gas sensor according to another configuration of the second embodiment has a hollow part 424-1 formed in the inside filter part 421. The hollow part 424-1 shown in FIG. 7 is smaller in volume than the hollow part 424 shown in FIG. 6.

As described above, it is possible to have the filter assembly of different configurations, as shown in FIG. 2, FIG. 3, FIG. 5, FIG. 6, and FIG. 7, for example.

The strength of the air filter and the air passage volume, through which outside atmosphere air is introduced into the inside of the gas sensor can be changed by changing the shape of the inside filter part in the filter assembly, for example, selecting one of the filter assemblies shown in FIG. 5 to FIG. 7. This can easily adjust and keep an optimum valance relationship between the air passage volume and the strength of the air filter.

For example, the air filter 42-1 shown in FIG. 5 has a superior air permeable capability when compared with the configuration of the air filter 42 shown in FIG. 3. On the contrary the air filter 42 shown in FIG. 3 has a high strength when compared with the air filter 42-1 shown in FIG. 5.

Further, the air filter 42-2 shown in FIG. 6 has a superior air permeable capability when compared with the configuration of the air filter 42-3 shown in FIG. 7. On the contrary, the air filter 42-3 shown in FIG. 7 has a high strength when compared with the configuration of the air filter 42-2 shown in FIG. 6.

As described above, changing the shape of the inside filter part in the air filter can take an optimum valance relationship between the air passage capability and the strength of the air filter.

### Third Embodiment

A description will be given of a gas sensor according to a third embodiment of the present invention with reference to FIG. 8 to FIG. 10

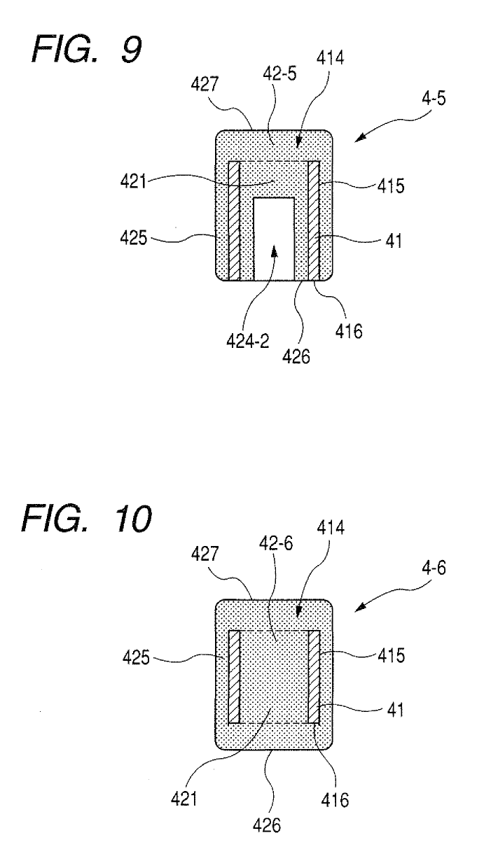

FIG. 8 is a sectional view of a filter assembly in the gas sensor according to the third embodiment. FIG. 9 is a sectional view of the filter assembly having another configuration in the gas sensor according to the third embodiment. FIG. 10 is a sectional view of the filter assembly having another configuration in the gas sensor according to the third embodiment.

The support member 41 has a straight shaped cylindrical member and is completely covered with the air filter 42. As shown in FIG. 8, a front end part 416 of the support member 41 and a front end part 426 of the air filter 42 have a same surface in the axial direction of the gas sensor.

Further, the filter assembly 4-5 shown in FIG. 9 and the filter assembly 4-4 shown in FIG. 8 have approximately the same outline. However, the air filter 42-5 in the filter assembly 4-5 shown in FIG. 9 has a hollow part 424-2 in the inside filter part 421.

Still further, in the filter assembly 4-6 shown in FIG. 10, because the support member 41 is completely embedded in the air filter 42-6, namely, no part of the support member 41 is exposed, it is possible to provide the gas sensor with superior manufacturing efficiency.

Other components of the filter assemblies shown in FIG. 8, FIG. 9, and FIG. 10 in the gas sensor according to the third embodiment of the present invention are the same as those of the first embodiment shown in FIG. 2 and FIG. 3.

In the filter assembly having the different configurations 4-4, 4-5, and 4-6 shown in FIG. 8, FIG. 9, and FIG. 10, because the support member 41 is embedded in the air filter, it is possible to easily make the filter assembly. This can provide the gas sensor with superior manufacturing efficiency.

The gas sensor having the filter assembly according to the third embodiment has the same actions and features of the gas sensor of the first embodiment.

### Comparison Example

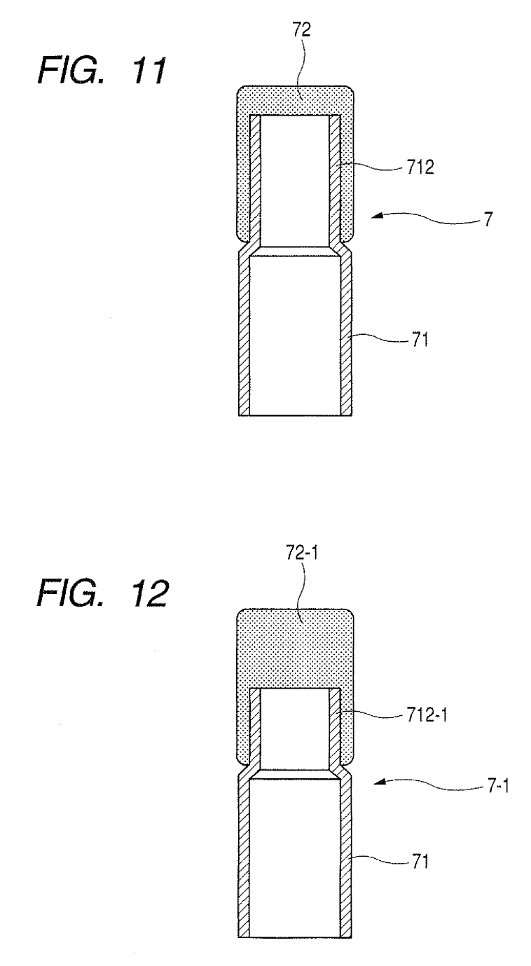

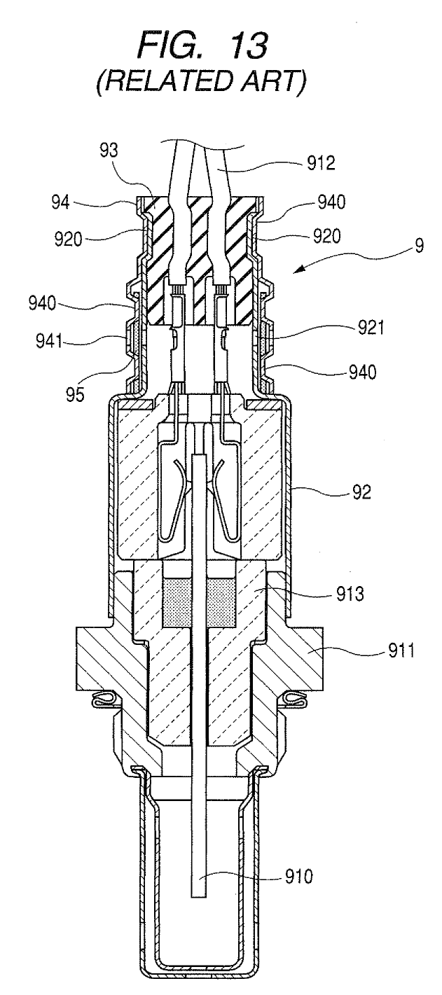

FIG. 11 is a sectional view of a filter assembly 7 in a gas sensor according to a comparison example. FIG, 12 is a sectional view of a filter assembly 7-1 having another configuration in the gas sensor as the comparison example.

As shown in FIG. 11 and FIG. 12, the filter assembly in the gas sensor according to the comparison example has no inside filter part. On the contrary, the filter assembly in the gas sensor according to the present invention has the inside filter part 421 shown in FIG. 5.

In the gas sensor of the comparison example, the same components of the gas sensor according to the first embodiment are designated by the same reference numbers and the explanation of them is omitted here.

Because the air filter 72 of the filter assembly 7 has no inside filter part when compared with the air filter 42 in the filter assembly 4 in the gas sensor according to the present invention, the air filter 72 shown in FIG. 11 has a smaller thickness than the air filter 42 in the gas sensor 1 according to the present invention. That is, the air filter in the gas sensor as the comparison example shown in FIG. 11 has a weak strength when compared with the strength of the air filter in the gas sensor according to the present invention.

Like the filter assembly 7 shown in FIG. 11, an air filter 72-1 in the filter assembly 7-1 as another comparison example shown in FIG. 12 has also no inside filter part. The filter assembly 7-1 shown in FIG. 12 is so formed that the thickness of the air filter 72-1 is larger than that of the air filter 72 shown in FIG. 11. Therefore the strength of the air filter 72-1 shown in FIG. 12 is stronger than that of the air filter 72 shown in FIG. 11.

When compared with the configuration of the filter assembly 7 shown in FIG. 11, pores in the air filter 72-1 in the filter assembly 7-1 shown in FIG. 12 are easily broken because a stress is directly applied to the base end side of the air filter 72-1 shown in FIG. 12 because the pressure is applied to the base end side of the air filter 72-1 from the elastic member (not shown). Accordingly, there is a possibility of not maintaining a desired air passage volume in the filter assembly 7-1 shown in FIG. 12.

On the contrary, the gas sensor according to the present invention, for example shown in FIG. 5, can easily keep a desired air passage volume, a desired air permeable function, and a required strength of the air filter because of having the inside filter part 421.

While specific embodiments of the present invention have been described in detail, it will be appreciated by those skilled in the art that various modifications and alternatives to those details could be developed in light of the overall teachings of the disclosure. Accordingly, the particular arrangements disclosed are meant to be illustrative only and not limited to the scope of the present invention which is to be given the full breadth of the following claims and all equivalent thereof.

What is claimed is:

1. A gas sensor comprising:
a sensor element configured to detect a concentration of a specific gas component contained in a target gas;
a housing accommodating the sensor element so that the sensor element is fitted to and supported by the housing;
lead wirings electrically connected to the sensor element;
an elastic member having a vertical hole in an axial direction of the gas sensor and lead wiring penetration holes, through which the lead wirings are inserted and disposed, and sealing the base end side of an atmosphere-side cover;
the atmosphere-side cover, which is disposed at a base end side of the housing, having fastening parts at which the elastic member is fastened toward a radius direction of the gas sensor;
a filter assembly comprised of a cylindrical shaped support member and an air filter fitted to the cylindrical shaped support member so that the air filter covers at least a base end side opening part of the cylindrical shaped support member and a part of an outer periphery surface continued to the base end side opening part of the cylindrical shaped support member, a part of the air filter covering the outer periphery surface of the support member is fitted and pressed between the elastic member and the support member, and a part of the air filter is disposed in the inside of the cylindrical shaped support member.

2. The gas sensor according to claim 1, wherein the air filter is fitted to the base end side of the cylindrical shaped support member.

3. The gas sensor according to claim 1, wherein the air filter and the cylindrical shaped support member are made in one assembly;

4. The gas sensor according to claim 2, wherein the air filter and the cylindrical shaped support member are made in one assembly.

5. The gas sensor according to claim 1, wherein at least a base end side part of the cylindrical shaped support member is embedded in the air filter,

6. The gas sensor according to claim 2, wherein at least a base end side part of the cylindrical shaped support member is embedded in the air filter.

7. The gas sensor according to claim 1, wherein the air filter has a hollow part formed in the inside thereof which faces the inside of the cylindrical shaped support member.

8. The gas sensor according to claim 2, wherein the air filter has a hollow part formed in the inside thereof which faces the inside of the cylindrical shaped support member.

9. The gas sensor according to claim 1, wherein the cylindrical shaped support member is embedded in the air filter.

10. The gas sensor according to claim 2, wherein the cylindrical shaped support member is embedded in the air filter.

11. The gas sensor according to claim 1, wherein the air filter is made of porous polytetrafluoroethylene.

12. The gas sensor according to claim 2, wherein the air filter is made of porous polytetrafluoroethylene.
